SEQUENCE LISTING (6 sequences)

SEQ ID NO: 1 - protein, 66 aa, Artificial Sequence
  modified IL-8 molecule
CQCIKTYSKPKHPKKIKELRVIESGPHCANTEIIVKLSDGRELCLDPKENWVQRVVEKFLKRAKKS

SEQ ID NO: 2 - protein, 62 aa, Artificial Sequence
  modified IL-8 molecule
KTYSKPXHPKXIKELRVIESGPHCANTEIIVKLSDGRELCLDPKENWVQRVVEKFLKRAXXS

SEQ ID NO: 3 - protein, 72 aa, Artificial Sequence
  modified IL-8 molecule
SAKELRCQCIKTYSKPFHPKFIKELRVIESGPHCANTEIIVKLSDGRELCLDPKENWVQRVVEKFLKRAENS

SEQ ID NO: 4 - protein, 66 aa, Artificial Sequence
  modified IL-8 molecule
CQCIKTYSKPXHPKXIKELRVIESGPHCANTEIIVKLSDGRELCLDPKENWVQRVVEKFLKRAXXS

SEQ ID NO: 5 - protein, 68 aa, Artificial Sequence
  modified IL-8 molecule
SAKELRKTYSKPXHPKXIKELRVIESGPHCANTEIIVKLSDGRELCLDPKENWVQRVVEKFLKRAXXS

SEQ ID NO: 6 - protein, 72 aa, Artificial Sequence
  modified IL-8 molecule
SAKELRCQCIKTYSKPXHPKXIKELRVIESGPHCANTEIIVKLSDGRELCLDPKENWVQRVVEKFLKRAXXS

(19) United States
(12) Patent Application Publication
(10) Pub. No.: US 2012/0288474 A1
(43) Pub. Date: Nov. 15, 2012

(54) COMPOSITION FOR TREATMENT OF CXCL8-MEDIATED LUNG INFLAMMATION

(75) Inventors: Andreas Kungl, Graz (AT); Jason Slingsby, Wien (AT); Tiziana Adage, Graz (AT); Angelika Rek, Graz (AT)

(73) Assignee: PROTAFFIN BIOTECHNOLOGIE AG, Graz (AT)

(21) Appl. No.: 13/395,128

(22) PCT Filed: Sep. 13, 2010
(86) PCT No.: PCT/EP2010/063389

§ 371 Date: May 23, 2012

(30) Foreign Application Priority Data
Nov. 6, 2009 (EP) 09175203.0
Mar. 1, 2010 (EP) 10155086.1

(51) Int. Cl. A61K 38/20; A61P 11/06; A61P 11/00; C07K 14/54; A61P 29/00

(52) U.S. Cl. 424/85.2; 530/300; 530/324

(57) ABSTRACT

The present invention provides a composition comprising a modified interleukin 8 (IL-8) having increased GAG binding affinity and further inhibited or down-regulated GPCR activity compared to the respective wild type IL-8 for use in preventing or treating lung inflammation with neutrophilic infiltration, for example for the prevention or treatment of chronic obstructive pulmonary disease, cystic fibrosis, severe asthma, bronchitis, broncheolitis, acute lung injury and acute respiratory distress syndrome.

The present invention relates to a new use of modified interleukin 8 (IL-8, CXCL8) having increased GAG binding affinity and further inhibited or down-regulated receptor binding activity compared to the respective wild type IL-8 for preventing or treating lung inflammation with neutrophilic infiltration, specifically for the prevention or treatment of CXCL8-mediated lung inflammation. Specifically the use of modified IL-8 as inhalant is provided.

### BACKGROUND OF THE INVENTION

Lung inflammatory diseases are of particular relevance in view of their pre-dominance in the human population and the lack of efficacious therapy. Specifically, lung diseases shown to have increased infiltration of neutrophils are chronic obstructive pulmonary disease, cystic fibrosis, chronic severe asthma and acute lung injury with its more severe form, acute respiratory distress syndrome.

Chronic obstructive pulmonary disease (COPD) is a progressive debilitating disease which is predicted to become the third leading cause of death worldwide by 2020 (Lopez et al. 1998). Cigarette smoke has been established as the most important etiological factor for its development, however only 15 to 20% of smokers develop COPD, suggesting that genetic component and other environmental factors play a role in the pathogenesis of the disease.

The inflammatory response observed in lungs of patients with COPD is complex and involves the activation of both innate and acquired immune responses; however it is clear that disease progression is dominated by leukocyte migration, the production of pro-inflammatory cytokines and chemokines and release of potentially destructive proteases (Kim et al. 2008).

In particular, neutrophils have been shown to be the most abundant inflammatory cells in lungs of COPD patients, both in sputum and bronchoalveolar lavage (BAL) samples (Nocker et al. 1996; Peleman et al. 1999). CXCL8 levels are significantly elevated in sputum and BAL of COPD patients at different stage of disease progression (between 10-15 fold increase vs. healthy) and correlate with disease severity and neutrophil presence (Yamamoto et al. 1997; Tanino et al. 2002), identifying CXCL8 as the key chemokine involved in neutrophil mobilization (Woolhouse et al. 2002). Further, elevated levels of CXCL8 are also present in sputum of COPD patients during exacerbations (Aaron et al. 2001; Spruit et al. 2003).

However, current therapies are acting more as supportive and symptomatic care, than having an anti-inflammatory activity. The drugs used for the management of COPD based on the recommendation of the World Health Organization and GOLD include short and long acting β2-agonist; short and long acting anticholinergic agents; methylxanthine, inhaled or systemic glucocosteroids (Pauwels et al., 2005; Lenfant and Khaltaev 2005). These therapies have some effects on controlling acute exacerbation, but treatment with traditional glucocorticosteroids results largely ineffective and fails in attenuate inflammation in patients with COPD (Culpitt et al. 1999; Fitzgerald et al. 2007), highlighting the need of the development of new anti-inflammatory strategies (Fabbri et al 2004).

Another lung disease characterized by neutrophilic inflammation is Cystic fibrosis (CF). Several studies have documented increased levels of CXCL-8 in BAL and sputum and increased expression of CXCL8 in bronchial glands of patients with CF (Nakamura et al. 1992; Tabary et al. 1998). Its potent neutrophil chemoattractant properties stimulate the influx of massive numbers of neutrophils in the airways (Chmiel et al. 2002). Bacterial infection are further increasing CXCL8 levels, driving more neutrophils infiltration into the lungs and creating a vicious circle difficult to interrupt and resulting in chronic lung inflammation. Acting on this vicious circle with treatments acting on CXCL8-induced inflammation, such as PA401, can result the most effective treatment for CF patients, which currently rely only on supportive therapy with bronchodilator and mucolytics or antibiotics.

About one in 10 asthmatics patients present the severe form of the disease, which frequently requires progressively higher doses of steroids in an attempt to control symptoms. Severe asthma is also associated with a much higher risk of illness and death than milder forms.

A strong association has now been established between neutrophilic inflammation and chronic severe asthma (Little et al. 2002; Wenzel et al. 1997, Jatakanon et al 1999, Ordonez at al. 2000, Kamath et al. 2005; Fahy 2009), childhood asthma (McDougall et al. 2006), asthma exacerbations (Fahy et al. 1995), corticosteroid resistant asthma (Green et al 2002), nocturnal asthma (Martin et al 1991), asthma in smokers (Chalmers et al. 2001) and occupational asthma (Anees et al.2002). It is now more and more recognised that chronic neutrophilic severe asthma presents a clear different clinical phenotype, rather than an increased presence in asthma symptoms and share features with COPD (The ENFUMOSA study group). Also in the case of chronic severe asthma, epithelial cell-derived CXCL8 is the most likely candidate as the predominant neutrophil chemoattractant (Lamblin et al. 1998; Ordonez et al. 2000), and potential candidate for the development of new anti-inflammatory therapies.

The aim of the present invention is therefore to provide a method for the prevention or treatment of lung inflammatory pathologies that are infiltrated with neutrophils.

### SUMMARY OF THE INVENTION

The invention is based on the discovery that modified interleukin 8 (IL-8) having increased GAG binding affinity and inhibited or down-regulated GPCR (G-protein coupled receptor, i.e. CXCR1 and CXCR2) activity compared to the respective wild type IL-8 can be used for the prevention and treatment of lung inflammation with neutrophilic infiltration. Especially in COPD and in COPD exacerbations, where increased levels of IL-8 are present and correlate with disease progression and severity (Yamamoto et al. 1997; Tanino et al. 2002), a therapeutic intervention targeting the key chemokine involved in neutrophil mobilization (Woolhouse et al. 2002) should provide beneficial anti-inflammatory activity. Moreover, current treatments for these patients rely on supportive and symptomatic care, while application of traditional glucocortico-steroids proved to be largely ineffective (Culpitt et al. 1999; Fitzgerald et al. 2007), highlighting the need of the development of new anti-inflammatory strategies (Fabbri et al. 2004, de Boer et al. 2007).

Although the use of modified IL-8 was already described briefly for the treatment of “normal” asthma lacking high levels of neutrophil infiltration in the lung, the successful use of said modified IL-8 molecules for the treatment and prevention of lung inflammation with neutrophil infiltration has not been shown or indicated before. The anti-asthma activity of said modified IL-8 molecules might result from non-specific or consecutive displacement of other, asthma-related, chemokines such as eotaxin.

The inhibited or down-regulated activity is at least a reduction or complete lack of neutrophil activation by GPCR activation. Although it has long been established that CXCL8-mediated lung inflammation or IL-8 induced neutrophil infiltration is significant in specific lung diseases, it has not been reported or suggested that said modified IL-8, a “protein-based GAG antagonist” would have such efficacy in the prevention or treatment of lung inflammatory diseases with neutrophilic infiltration.

Subject matter of the present invention is therefore to provide a modified IL-8 having increased GAG binding affinity and inhibited or down-regulated GPCR activity compared to the respective wild type IL-8 for use for the prevention or treatment of lung inflammatory diseases with neutrophilic infiltration in individuals.

### FIGURES

FIG. 1 Dose-response effect of PA401 on total cell infiltrates in bronchoalveolar lavages of mice instilled with LPS.

FIG. 2 Dose-response effect of PA401 on neutrophils number in cytospin of bronchoalveolar lavages of mice instilled with LPS.

FIG. 3 Dose-response effect of PA401 on lymphocytes number in cytospin of bronchoalveolar lavages of mice instilled with LPS.

FIG. 4 Dose-response effect of PA401 on total cell infiltrates in bronchoalveolar lavages of mice aerosolized with LPS.

FIG. 5 Dose-response effect of PA401 on neutrophils number in cytospin of bronchoalveolar lavages of mice aerosolized with LPS.

FIG. 6 Dose-response effect of PA401 (FIG. 6a) and Roflumilast (FIG. 6b) on total cell infiltrates in bronchoalveolar lavages of mice exposed for 4 days to cigarette smoke.

FIG. 7 Dose-response effect of PA401 (FIG. 7a) and Roflumilast (FIG. 7b) on neutrophil infiltrates in bronchoalveolar lavages of mice exposed for 4 days to cigarette smoke.

FIG. 8 Dose-response effect of PA401 (FIG. 8a) and Roflumilast (FIG. 8b) on macrophage infiltrates in bronchoalveolar lavages of mice exposed for 4 days to cigarette smoke.

FIG. 9 Dose-response effect of PA401 (FIG. 9a) and Roflumilast (FIG. 9b) on epithelial cell infiltrates in bronchoalveolar lavages of mice exposed for 4 days to cigarette smoke.

FIG. 10 Dose-response effect of PA401 (FIG. 10a) and Roflumilast (FIG. 10b) on lymphocyte infiltrates in bronchoalveolar lavages of mice exposed for 4 days to cigarette smoke.

FIG. 11: Effect on total cell infiltrates after twice a day, daily and every other day administration of PA401 (FIG. 11a) and and comparison with Roflumilast (FIG. 11b).

FIG. 12: Reduction of neutrophils at all treatment frequencies by PA401 (FIG. 12a) and comparison with Roflumilast (FIG. 12b).

FIG. 13: Reduction of epithelial cells at all treatment frequencies by PA401 (FIG. 13a) and comparison with Roflumilast (FIG. 13b).

FIG. 14: Loss of significant reduction in lymphocyte obtained with b.i.d. and q.d. administration when PA401 was administered every other day (FIG. 14a) and comparison with Roflumilast (FIG. 14b).

FIG. 15: Loss of significant reduction in macrophage obtained with b.i.d. and q.d. administration when PA401 (FIG. 15a) was administered every other day. Comparison with Roflumilast (FIG. 15b).

FIG. 16: Effect of PA401 (FIG. 16a) on total cell infiltrates observed after 400 and 40 μg/kg administration. Comparison with Roflumilast (FIG. 16b).

FIG. 17: Reduction in neutrophil numbers in BAL (PA401, FIG. 17a and Roflumilast, FIG. 17b)

FIG. 18: Reduction in epithelial cell numbers in BAL (PA401, FIG. 18a and Roflumilast, FIG. 18b)

FIG. 19: Reduction in lymphocyte numbers in BAL (PA401, FIG. 19a and Roflumilast, FIG. 19b)

FIG. 20: Reduction in macrophage numbers in BAL (PA401, FIG. 20a and Roflumilast, FIG. 20b)

FIG. 21 Dose-response activity on PA401 intra-tracheal administration 1 hour before (−1 h) and 1 hour after (+1 h) LPS aerosol exposure on total cell count in the bronchoalveolar lavage collected 8 hours post LPS. ANOVA followed by Dunnett's test: *p<0.05;**p<0.01 versus vehicle treated animals.

FIG. 22 Dose-response activity on PA401 intra-tracheal administration 1 hour before (−1 h) and 1 hour after (+1 h) LPS aerosol exposure on neutrophils count in the bronchoalveolar lavage collected 8 hours post LPS. ANOVA followed by Dunnett's test: *p<0.05;**p<0.01 versus vehicle treated animals.

### DETAILED DESCRIPTION OF THE INVENTION

CXCL8-mediated lung inflammation can lead to neutrophil infiltration in the lung of patients.

The present invention covers a modified interleukin 8 (IL-8) having increased GAG binding affinity and further inhibited or down-regulated GPCR activity compared to the respective wild type IL-8 for the use for the prevention or treatment of lung inflammation with neutrophilic infiltration. The modified IL-8 molecule as used in the present invention for the treatment of lung inflammation with neutrophil infiltration is modified in the GAG (glycosaminoglycan) binding region leading to increased affinity towards GAG, the IL-8-specific GAG ligand. Modification can be either in the naturally occurring GAG binding region or, alternatively, a new GAG binding region can be introduced in said molecule resulting in increased affinity towards GAG. By substituting at least one naturally occurring amino acid against an amino acid, preferably a basic or electron donating amino acid, and/or substituting at least one bulky and/or acidic amino acid in the GAG binding region, an artifical and/or improved GAG binding site is introduced in said protein. By this means, an overall more electronegative molecular character can be introduced into the chemokine.

The main purpose is to increase the relative amount of basic or electron donating amino acids, preferably Arg, Lys, His, Asn and/or Gln, compared to the total amount of amino acids in said site, whereby the resulting GAG binding site should preferably comprise at least 3 basic amino acids, still preferred at least 4, most preferred at least 5 amino acids. This leads to a chemokine-based GAG antagonist competing wtIL-8 off from its HSPG co-receptor.

According to a specific embodiment the GAG binding site is a C-terminal alpha helix which is modified to increase GAG binding affinity.

The term “bulky amino acid” refers to amino acids with long or sterically interfering side chains; these are in particular Trp, Ile, Leu, Phe, Tyr. Preferably, the GAG binding site on the chemokine is free of bulky amino acids to allow optimal induced fit by the GAG ligand. Advantageously, positions 17, 21, 70, and/or 71 in IL-8 are substituted by Arg, Lys, His, Asn and/or Gln. Most preferred, all four positions 17, 21, 70 and 71 of IL-8 are substituted by Arg, Lys, His, Asn and/or Gln, preferably by Lys.

The modified IL-8 as used in the present invention comprises also an inhibited or down-regulated receptor binding region, specifically the GPCR (G-protein coupled receptor) binding. Inactivation by protein engineering according to the present invention therefore leads to an IL-8 molecule which is reduced in promoting neutrophil activation or incapable of promoting neutrophil activation.

This means for the entire approach, that on the one hand the GAG binding affinity is higher than in the wild-type GAG binding protein, so that the modified protein will to a large extent bind to the GAG instead of the wild-type protein. On the other hand, the GPCR activity of the wild-type protein which mainly occurs when the protein is bound to GAG, is inhibited or down-regulated, since the modified protein will not carry out this specific activity or carries out this activity to a lesser extent.

The receptor binding region can be modified by deletion, insertion, and/or substitution, for example with alanine, a sterically and/or electrostatically similar residue. It is possible to either delete or insert or substitute at least one amino acid in a receptor binding region.

In the used modified IL-8 said GPCR binding region is located within the first 10 N-terminal amino acids. The first N-terminal amino acids are involved in leukocyte activation, whereby in particular Glu-4, Leu-5 and Arg-6 were identified to be essential for receptor binding and activation. Therefore, either these three or even up to the first 10 N-terminal amino acids can be substituted or deleted in order to inhibit or down-regulate the receptor binding and activation.

For example, the modified IL-8 can have the first 6 N-terminal amino acids deleted. As mentioned above, this mutant will not or to a lesser extent bind and activate leukocytes and/or promote neutrophil activation, so that it is particularly suitable for the treatment of organ transplant rejection.

Preferably, the modified IL-8 is selected from the group consisting of del6F17RE70KN71R, del6F17RE70RN71K, del6E70KN71K, del6F17RE70RN71K, and del6F17KF21KE70KN71K.

The amino acid sequence of the modified IL 8 molecule is preferably described by the general formula:

(X1)ₙ(X2)ₘ KTYSKP(X3)HPK (X4)IKELRVIES GPHCANTEII
VKLSDGRELC LDPKENWVQR VVEKFLKRA(X5)(X6)S

wherein X1 is of amino acid sequence SAKELR,

wherein X2 is of amino acid sequence CQCI,

wherein X3 is selected of the group consisting of R, K, H, N and/or Q, preferably it is R,

wherein X4 is selected of the group consisting of R, K, H, N and/or Q,

wherein X5 is selected of the group consisting of R, K, H, N and/or Q, preferably it is K,

wherein X6 is selected of the group consisting of R, K, H, N and/or Q, preferably it is K,

and wherein n and/or m can be either 0 or 1

In a preferred embodiment the sequence of the modified IL-8 molecule is as follows:

[symbol]

CQCI KTYSKPFHPK FIKELRVIES GPHCANTEII
VKLSDGRELC LDPKENWVQR VVEKFLKRAE NS
wherein
the first 6 amino acids (SAKELR) are deleted.

Preferably, the modified IL-8 is similar or identical to modified IL-8 as disclosed in WO 05/054285.

The administration of the composition may be by intravenous, intramuscular or subcutaneous route. Other routes of administration, which may establish the desired blood levels of the respective ingredients such as systemic administration or inhalation, are also comprised.

Specifically it has been shown that local delivery to the lung, preferably inhalation or intratracheal administration is an advantageous administration mode. Therefore the modified IL-8 can be formulated as inhalant and can be administered by an inhalation system as known in the art. The modified IL-8 can be formulated as liquid, aerosol or powder.

The medicament comprising the composition according to the invention can be formulated together with a pharmaceutically acceptable carrier.

“Pharmaceutically acceptable” is meant to encompass any carrier, which does not interfere with the effectiveness of the biological activity of the active ingredient and that is not toxic to the host to which is administered. For example, for parenteral administration, the modified IL-8 may be formulated in unit dosage form for injection in vehicles such as saline, dextrose solution, serum albumin and Ringer's solution. Besides the pharmaceutically acceptable carrier also minor amounts of additives, such as stabilisers, excipients, buffers and preservatives can be included.

The modified IL-8 comprising composition is used to prepare a medicament to prevent or treat any lung inflammatory diseases which are characterized by neutrophil infiltration. More specifically these diseases can be for example chronic obstructive pulmonary disease, cystic fibrosis, severe asthma, bronchitis, broncheolitis, acute lung injury and acute respiratory distress syndrome.

Specifically, the modified IL-8 is used for therapy of COPD.

As an alternative embodiment, prevention or treatment of neutrophilic asthma or exacerbations is specifically covered using modified IL-8 according to the description.

According to the present invention also any lung inflammation can be treated or prevented which is induced by LPS inhalation since LPS is one of the major factors inducing IL-8 expression (Chemokines and chemokine receptors in infectious diseases. (Mahalingam S, Karupiah G., Immunol Cell Biol. 1999 December; 77(6):469-75). LPS is a component of the walls in Gram-negative bacteria and is therefore present when gram negative bacterial infections occur or is present in air pollutant and in the tobacco leaves (so in cigarette smoke as well).

Alternatively, a method for treatment of lung inflammation with neutrophilic infiltration in a subject in need thereof comprising administering to the subject a therapeutically effective amount of modified IL-8 is covered by the invention. Specifically, the administration is by inhalation or by intratracheal administration.

Chronic Obstructive Pulmonary Disease (COPD).

In particular, neutrophils have been shown to be the most abundant inflammatory cell in lungs of COPD patients, both in sputum and bronchoalveolar lavage (BAL) samples (Nocker et al. 1996; Peleman et al. 1999). Cigarette smoke is considered to be responsible for elevation of circulating neutrophils, probably due to increased mobilization from the bone marrow (Cowburn et al. 2008), and their sequestration to the lung capillaries were they exit the pulmonary circulation. This feature is peculiar for the pulmonary circulation, since in the systemic circulation neutrophils exit at the level of postcapillary venules.

Neutrophils are then mobilized to the bronchial walls and lung parenchyma (Peleman at al. 1999; Kim et al. 2008). Activation of neutrophils with the subsequent release of reactive oxygen species and elastase is considered the leading cause for the development of lung damage and chronic dysfunction. Indeed, blood neutrophilia has been since long correlated with the rate of decline in lung functions measured in term of forced expiratory volume (FEV; Sparrow et al. 1984).

CXCL8 levels are significantly elevated in sputum and BAL of COPD patients at different stage of disease progression (between 10-15 fold increase vs. healthy) and correlate with disease severity and neutrophil presence (Yamamoto et al. 1997; Tanino et al. 2002), identifying CXCL8 as the key chemokine involved in neutrophil mobilization (Woolhouse et al. 2002). Further, elevated levels of CXCL8 are also present in sputum of COPD patients during exacerbations (Aaron et al. 2001; Spruit et al. 2003).

Cystic Fibrosis

Cystic fibrosis (CF) is a severe monogenic disorder of ion transport in exocrine glands, with different mutations in the CF transmembrane conductance regulator (CFTR) gene leading to impaired epithelial chloride secretion (Riordan 1989; Ratjen 2009). Dehydration and plugging of mucous secretions in the ducts of exocrine glands predispose to multi-organ clinical manifestations, particularly in the gastrointestinal, hepatobiliary, reproductive and respiratory tracts.

Chronic bacterial infections and inflammation of the lung are the main causes of morbidity and mortality in CF patients (Ratjen 2006). With increasing age, CF patients develop airway obstruction and many of these patients also suffer from airway hyper-responsiveness and asthma-like symptoms.

Many inflammatory cytokines are produced in the airways in CF patients (Sagel et al. 2002). Several studies have documented increased levels of CXCL-8 in BAL and sputum and increased expression of CXCL8 in bronchial glands of patients with CF (Nakamura et al. 1992; Tabary et al. 1998). Its potent neutrophil chemoattractant properties stimulate the influx of massive numbers of neutrophils in the airways (Chmiel et al. 2002). Moreover neutrophils from CF children display a higher migratory responsiveness to CXCL8 in vitro compared to those of non CF, suggesting that persistent elevated CXCL8 levels can “prime” CF neutrophils (Brennan et al. 2001).

In a recent study on in vitro co-culture of CF neutrophils and bronchial epithelial cells bearing the CFTR mutation, it is suggested that in CF patients a high number of non-apoptotic neutrophils adherent on airway epithelium and associated with elevated CXCL8 levels may contribute to sustained and exaggerated inflammatory response in the airways (Tabary et al. 2006).

Indeed, the Na—Cl imbalance seems to be the first cause of CXCL8 increased production and subsequent neutrophil infiltration. Bacterial infection are further increasing CXCL8 levels, driving more neutrophils infiltration into the lungs and creating a vicious circle difficult to interrupt and resulting in chronic lung inflammation. Acting on this vicious circle can result the most effective treatment for CF patients, which currently rely only on supportive therapy or antibiotics.

### Severe Asthma

Eosinophil inflammation has for long been considered the most distinctive pathological hallmark of asthma (Bousquet 1990). However, eosinophil inflammation is present in the airways of only 50% of asthmatic patients (Douwes et al. 2002), and often not observed in asthma exacerbations.

A strong association has now been established between neutrophilic inflammation and severe asthma (Little et al. 2002; Wenzel et al. 1997, Jatakanon et al 1999, Ordonez at al. 2000, Kamath et al. 2005; Fahy 2009), childhood asthma (McDouglas et al. 2006), asthma exacerbations (Fahy et al. 1995), corticosteroid resistant asthma (Green et al 2002), nocturnal asthma (Martin et al 1991), asthma in smokers (Chalmers et al. 2001) and occupational asthma (Anees et al.2002).

During an acute asthma attack, eosinophils and neutrophils are coexisting (Wenzel et al 1999), but it is suggested that neutrophils are becoming the predominant cell population over time. Their presence in the airways is in proportion to disease severity and progression (Wenzel et al 1997) and is associated to airflow obstruction and reduced lung function (Shaw et al. 2007). Neutrophils are also the main leukocyte population observed in a very severe and often lethal form of asthma characterized by sudden-onset (sudden-onset asthma; Sur et al. 1993).

Acute Lung Injury (ALI) and Acute Respiratory Distress Syndrome (ARSD)

Acute lung injury (ALI) and its more severe form, acute respiratory distress syndrome (ARDS), represent two different stages of the same disease, characterized by acute lung inflammation with enhanced vascular permeability and lung oedema formation of increasing severity (Bernard et al. 1994). The pathology results in a very high mortality rate (about 40%) with no trend in decreasing in the last decade, despite improved intensive care intervention (Puha et al. 2009).

The involvement of neutrophils in ALI and ARDS has been documented by several groups (Ware et al 2000; Abraham 2003) and increased level of neutrophils in BAL and its correlation with increased CXCL8 levels has been reported (Aggarwal et al. 2000). Moreover CXCL8 elevated levels in BAL have been correlated with ARDS development on at-risk patients (Donnelly et al. 1993; Reid et al. 1995).

In the lung, glycosaminoglycans (GAGs) are the main component of the non-fibrillar compartment of the interstitium, and are located in the sub-epithelial tissue and in the bronchial walls, as well as in the airways secretions. Their presence is essential to regulate hydration and water homeostasis, maintain tissue structure, and modulate inflammatory responses (e.g. rev. Souza-Fernandes et al. 2006).

Compared to other human pathologies not a lot of literature is available supporting the involvement of GAGs in mediating chemokine actions in pulmonary diseases.

All four classes of glycosaminoglycans, including heparin/heparan sulphate, chondroitin/dermatan sulfate, keratin sulfate, and hyaluronan are present in normal lungs. Heparan sulfate has been reported as the predominant form (˜40%), followed by chondroitin/dermatan sulphate (˜31%) and to minor extent to hyaluronan (˜14%) and keratin sulfate (Frevert et al. 2003).

The foregoing description will be more fully understood with reference to the following examples. Such examples are, however, merely representative of methods of practicing one or more embodiments of the present invention and should not be read as limiting the scope of invention.

### EXAMPLES

Considering that in most, if not all lung pathologies an alteration of the lung vascular permeability and of the matrix is observed, systemic administration of PA401 (del6F17KF21KE70KN71K IL-8 mutant) should be appropriate to reach adequate levels of PA401 at both lung venule and epithelial level.

### Example 1

PA401 Effects on LPS-Induced Acute Lung Inflammation Models in Mice.

A variety of stimuli induce neutrophil migration into the lung. Among the most frequently used and best characterized inflammatory inducer is the endotoxin of Gram-negative bacteria (lipopolysaccharide: LPS).

LPS instilled intranasal, or aerosolized induces a dose and time dependent neutrophil infiltration in the lung vasculature, interstitium and BAL (Reutershan et al. 2005), with peak levels reached between 4-8 hours post challenge, and remaining significantly above baseline for up to 24 hours in mice.

The LPS dose administered varies based on the LPS serotype, the method of application and the strain of the mice used, with significant BAL neutrophilia reported for doses of LPS as low as 0.1 μg/mouse and up to 800 μg/mouse.

LPS inhalation is able to induce lung neutrophil infiltration across species (e.g. mice and rats, Chapman et al.2007; guinea pigs; Wu et al. 2002; rabbits, Smith et al. 2008; sheep, Waerhaug et al. 2009; horses, van den Hoven et al. 2006; dogs, Koshika et al. 2001). Inhalation of 1 to 100 μg of LPS in healthy volunteers is regarded as robust and reliable model for acute lung inflammation (Mans et al. 2005, Kitz et al. 2008) as well as chronic obstructive lung disease exacerbation (Kharitonov et al, 2007).

To assess potential of PA401 as anti-inflammatory in models of acute lung neutrophilia, a dose-response study with subcutaneous administration of PA401 at doses of 4, 40, 200 and 400 μg/Kg in C57BL/6J female mice intranasal instilled with LPS (0.3 μg/mouse, serotype pseudomonas aeruginosa) was performed. Sham instilled (saline) and Dexamethasone 3 mg/kg s.c. (administered at t=−1 h before LPS instillation) treated animals were used as controls. Bronchoalveolar lavage (BAL) was performed 4 h post LPS instillation and total cell count on BAL samples, as well as differential cell count on BAL cytospin were measured.

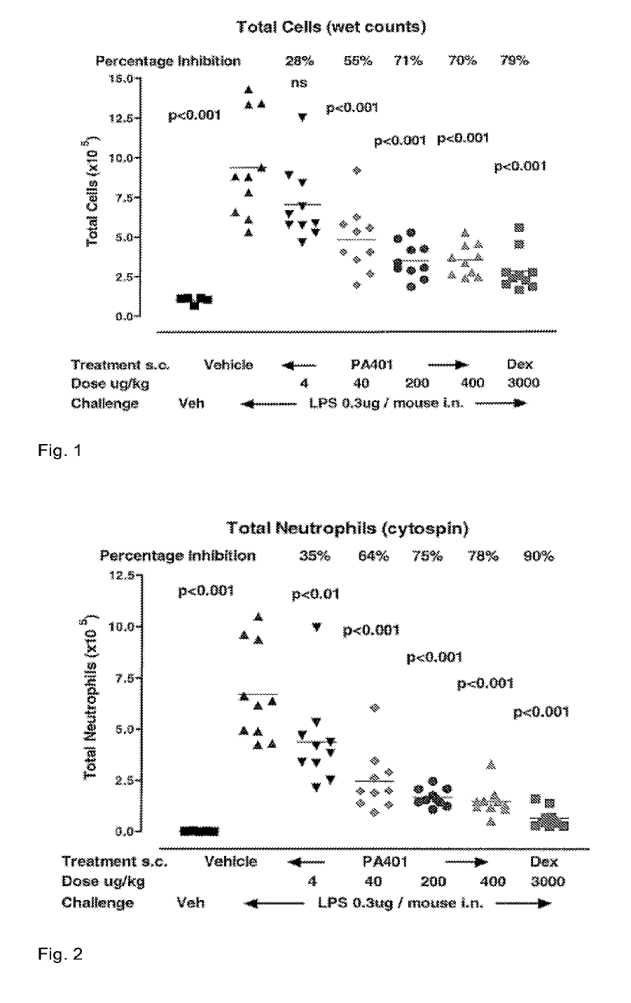

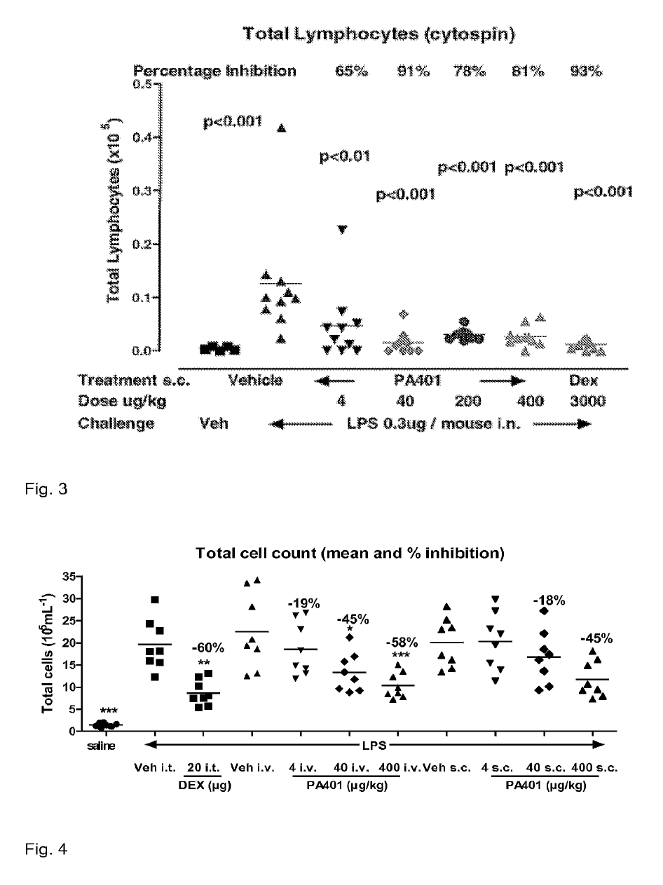

PA401 induced a dose-dependent reduction in the total number of cells infiltrated in lung as assessed in the BAL fluid (FIG. 1). The effect was due to a significant reduction in the number of neutrophils (FIG. 2) and lymphocytes (FIG. 3), with significant effect for dose of PA401 as low as 4 μg/kg. Surprisingly, the inhibition of cell infiltration in the BAL obtained with PA401 at the doses of 200 and 400 μg/kg was comparable to that obtained with the treatment with a high dose of Dexamethasone (3 mg/kg). This study was conducted at Argenta Discovery Ltd, UK.

### Example 2

A second study was performed using a slightly different model, which imply a different mouse strain and gender (male Balb/c instead of C57BL/6) a different LPS strain and serotype (Salmonella enterica instead of E. Coli) and a different LPS administration (aerosol—3.5 mg/7 mL over 30 min—instead of intranasal). PA401 doses of 4, 40 and 400 μg/kg were administered either by s.c. or i.v. route at t=−5 and t=+3 h from LPS exposure. BAL total and differential cell count were evaluated at t=8 h, a later time point compared to the previous study.

Saline aerosolized mice and mice receiving an intra tracheal administration of Dexamethasone (20 μg/20 ∥l/mouse at t=−1 h) were used as control.

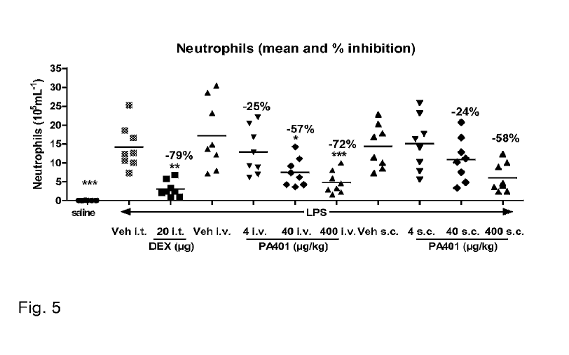

Also in this case PA401 induced a highly significant reduction in the number of total cells in the BAL (FIG. 4), due to reduction in neutrophils count (FIG. 5). The activity of PA401 was more significant when administered by intravenous than by subcutaneous route, reaching the same inhibitory effects of intra tracheal administration of dexamethasone. The study was performed at Pneumolabs Ltd, UK.

These studies demonstrate strong activity of PA401 administered by subcutaneous or intravenous route in 2 acute lung neutrophilic inflammatory animal models resembling human ALI/ARDS and COPD exacerbations. The effect was obtained independently of gender or genetic background of the animals; the LPS serotype and after both intranasal instillation and aerosol exposure.

### Example 3

PA401 Effects in an Acute Model of Cigarette Smoke Induced Lung Inflammation.

Acute exposure of mice to cigarette smoke leads to lung responses that, at least in part, mimic the lung inflammation observed in COPD patients. Different mouse strains present variable degree of lung inflammation following acute cigarette smoke exposure (Guerrassimov et al. 2004, Vlahos et al. 2006). This genetic variability in the response in mice appears quite representative of the variable susceptibility to develop COPD among human smokers, and therefore this model is considered the most relevant to model the human pathology.

Lung inflammation was induced in C57BL/6J female mice (a susceptible strain) by exposure to cigarette smoke of 4 to 6 cigarette over a four-day period. Dose response activity of subcutaneous PA401 treatment at the doses of 4, 40 and 400 μg/kg administered at t=+30 min and t=+6 h from smoke exposure, on cell infiltrates on bronchoalveolar lavages was evaluated 24 h after last cigarette smoke exposure. Air exposed, and Roflumilast (5 mg/kg oral) treated animals serve as controls.

Also in this animal model of COPD, PA401 dose dependently inhibited the cigarette smoke induced increased in total cell number recovered in the BAL.

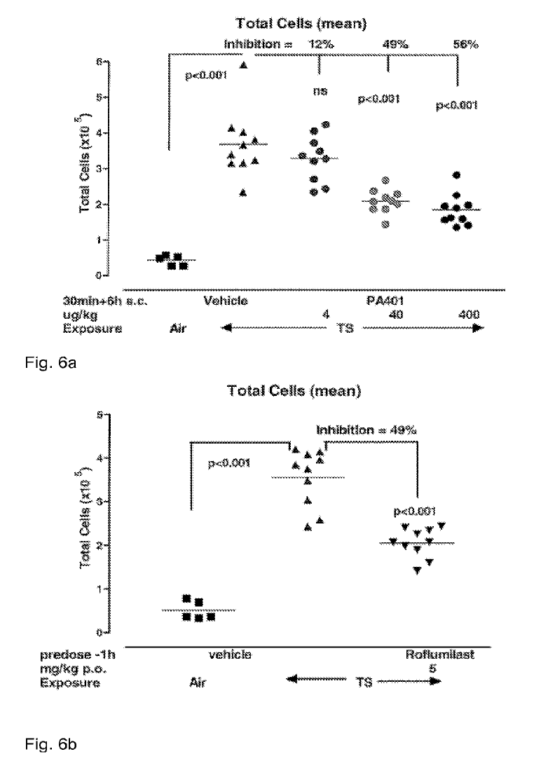

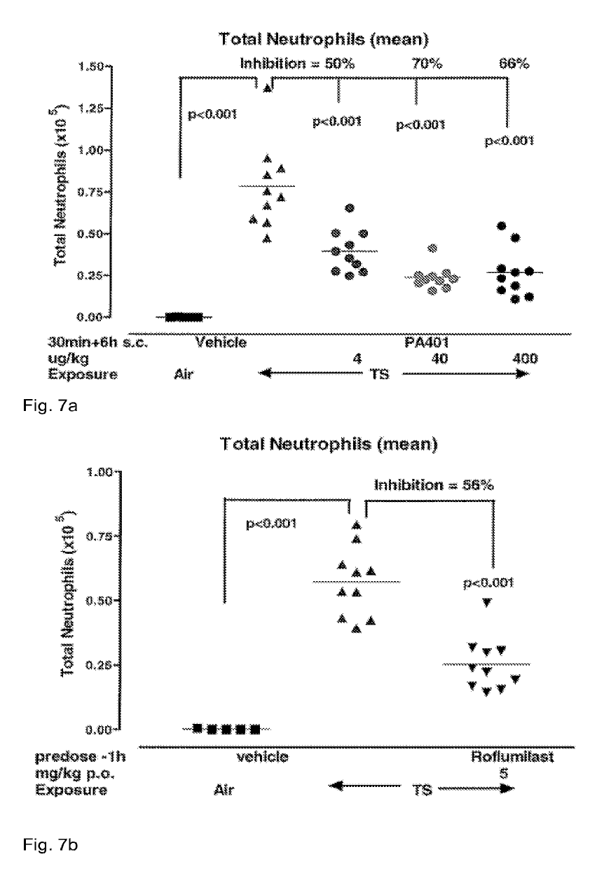

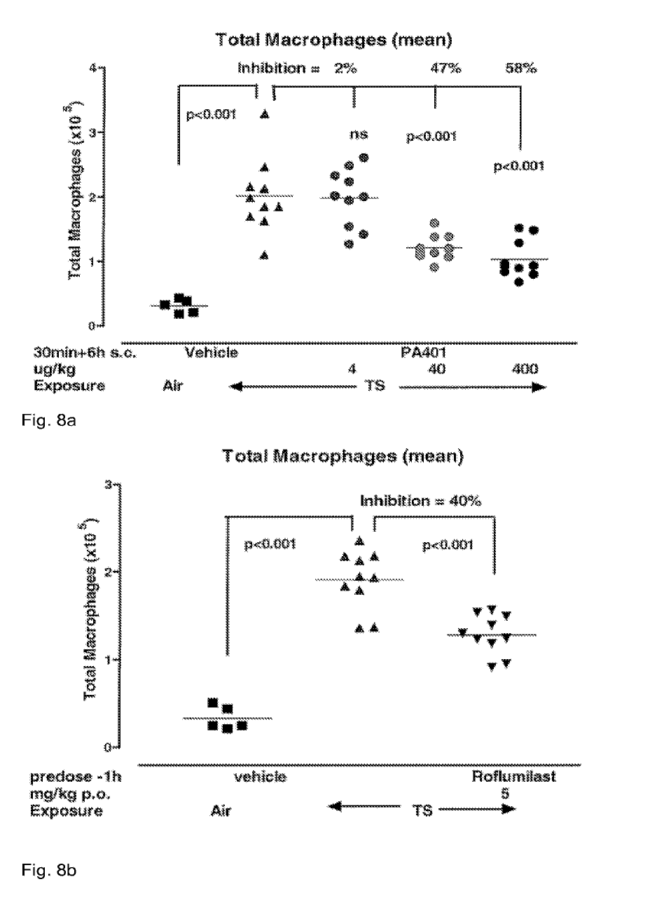

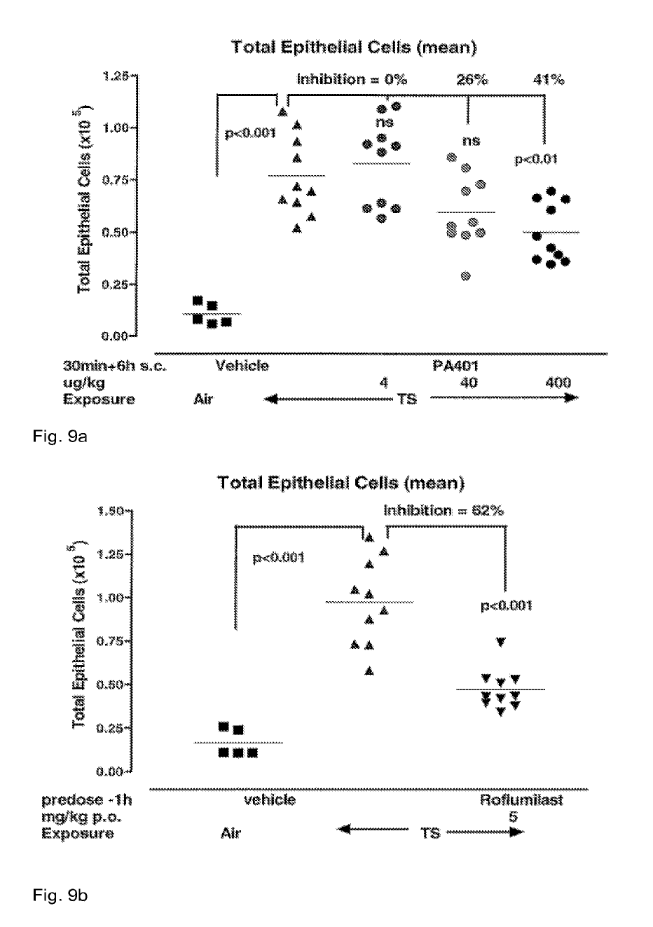

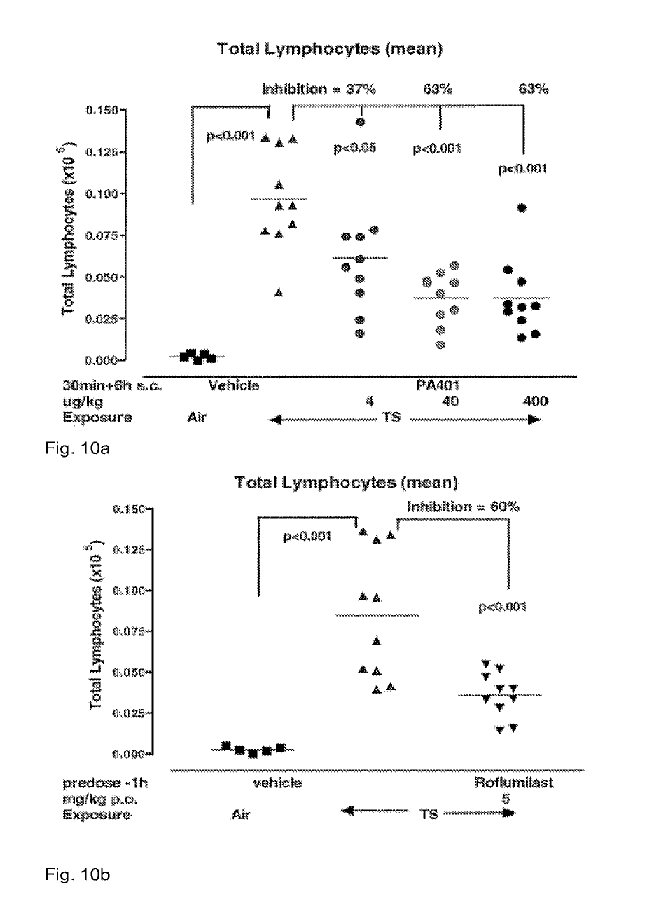

Highly significant effect on total cell infiltrates was observed at 40 and 400 μg/kg. (FIG. 6). At the highest dose used, PA401 significantly reduced neutrophil (FIG. 7), macrophage (FIG. 8), epithelial cells (FIG. 9) as well as lymphocyte (FIG. 10) numbers in BAL. Significant effect on most of these cell subtypes were observed also for the other two doses used in the study. The inhibitory effect of PA401 400 μg/kg was comparable of that obtained with Roflumilast 5 mg/kg. This study was performed at Argenta Discovery Ltd, UK.

PA401

This study demonstrates activity of PA401 on mixed cell infiltration induced by 4-day repeated exposure to cigarette smoke, and animal model predictive of anti-inflammatory activities in COPD patients.

### Example 4

PA401 Effects in a Sub-Chronic Model of Cigarette Smoke Induced Lung Inflammation.

In this study lung inflammation was induced in C57BL/6J female mice by exposure to cigarette smoke of 4 to 6 cigarettes over an eleven-day period. Dose frequency activity of subcutaneous PA401 treatment at the optimal dose of 400 μg/kg, based on the study in example 3, administered at t=+30 min and t=+6 h from smoke exposure (twice a day: b.i.d.), once daily at +3 h (q.d) and every other day (q.o.d) at +3 h, on cell infiltrates on bronchoalveolar lavages was evaluated 24 h after last cigarette smoke exposure. Air exposed, and Roflumilast (5 mg/kg oral) treated animals serve as controls.

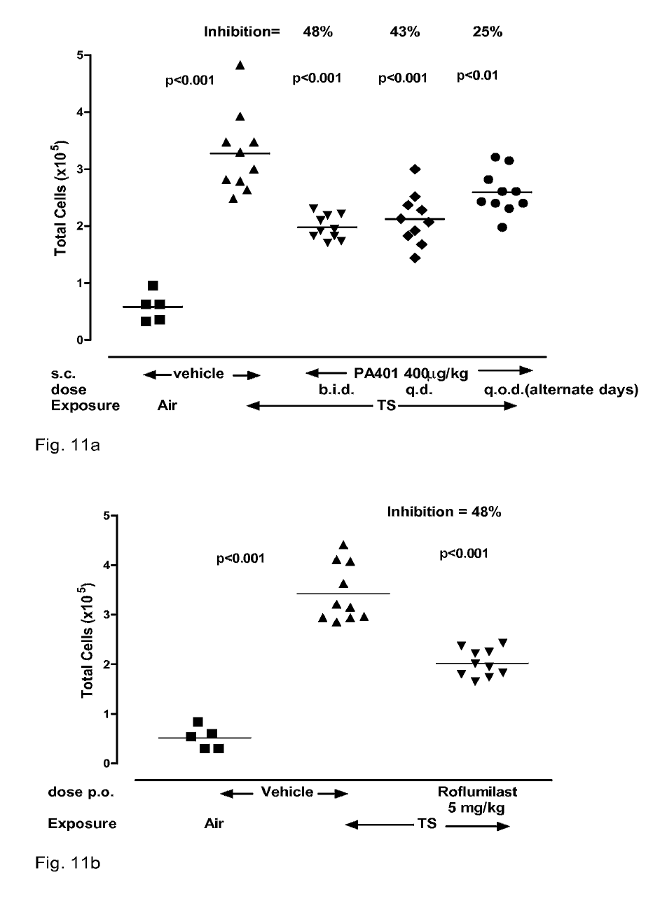

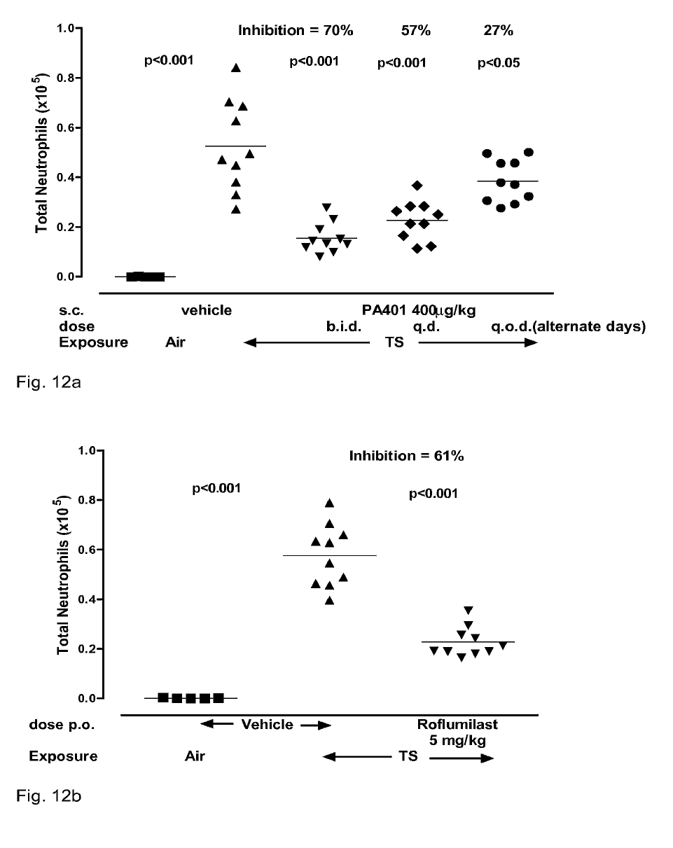

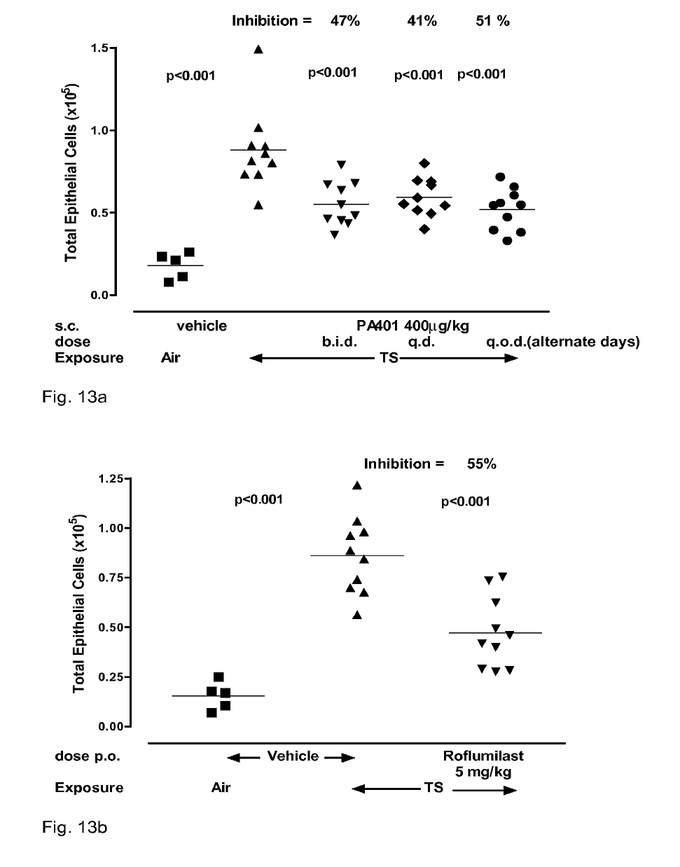

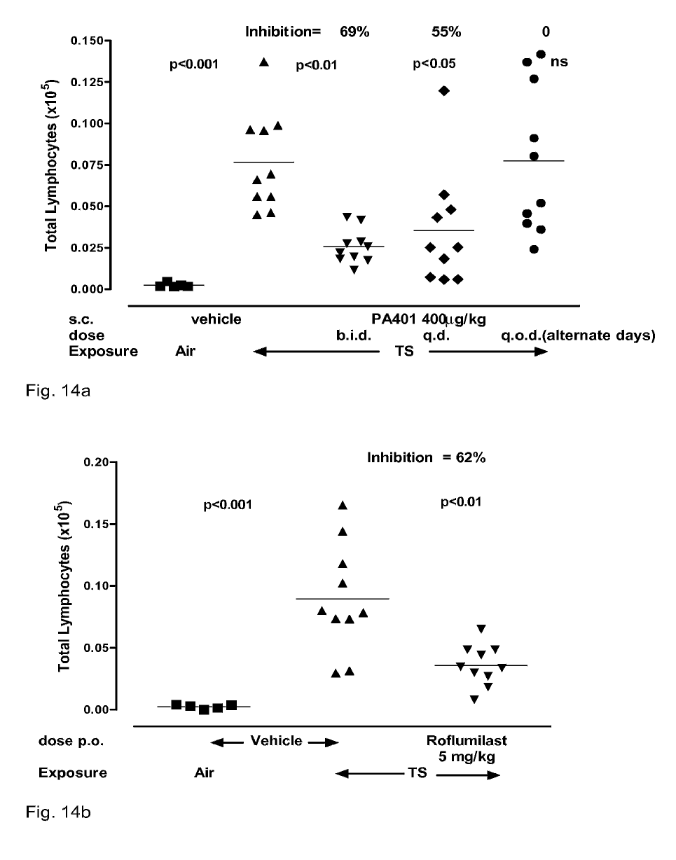

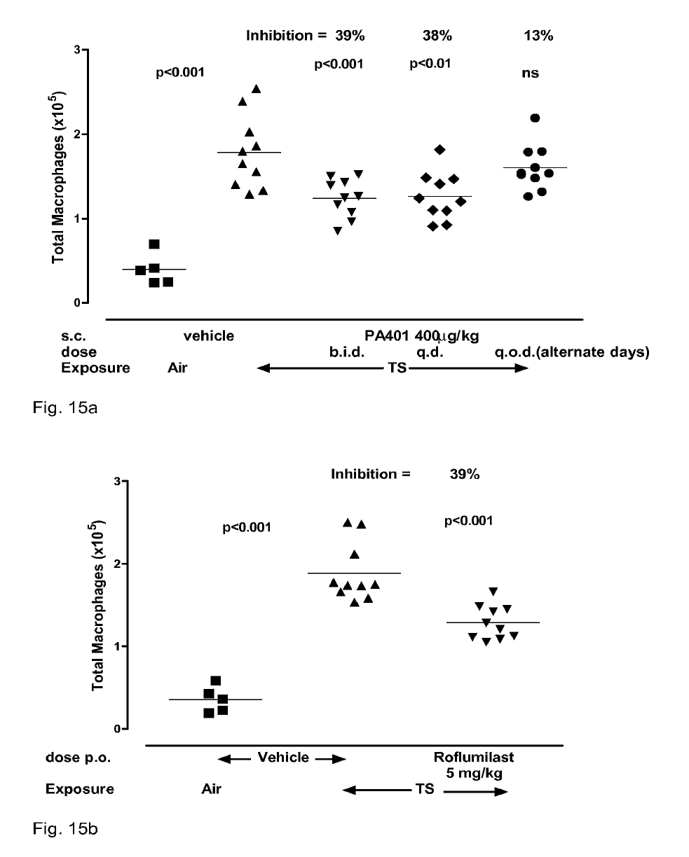

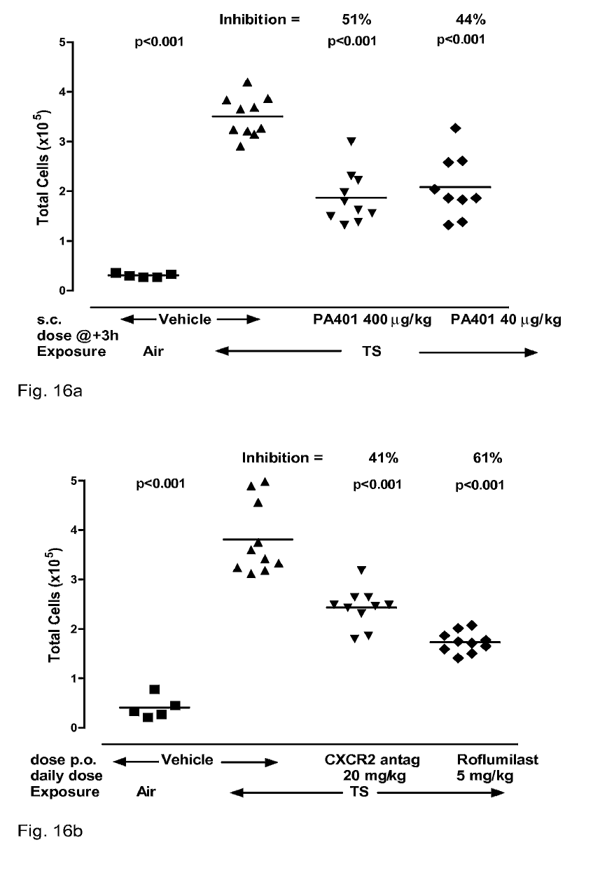

Significant effect on total cell infiltrates was observed after twice a day, daily and every other day administration (FIG. 11). At all the treatment frequency used, PA401 significantly reduced neutrophil (FIG. 12) and epithelial cells (FIG. 13) numbers in BAL, while the significant reduction in lymphocyte (FIG. 14) and macrophage (FIG. 15) obtained with b.i.d. and q.d. administration was lost when PA401 was administered every other day. The inhibitory effect of PA401 administered twice and once a day were comparable of that obtained with Roflumilast 5 mg/kg. This study was performed at Argenta Discovery Ltd, UK.

PA401

This study demonstrates that PA401 administration at the dose of 400 μg/kg subcutaneously twice a day and once a day have comparable activity on mixed cell infiltration induced by 11-day repeated exposure to cigarette smoke.

### Example 5

PA401 Effects in a Sub-Chronic Model of Cigarette Smoke Induced Lung Inflammation.

In this study lung inflammation was induced in C57BL/6J female mice by exposure to cigarette smoke of 4 to 6 cigarettes over an eleven-day period. PA401 at the dose of 400 μg/kg daily subcutaneous was compared to the dose of 40 μg/kg daily subcutaneous. Treatment was performed at t+3 h and effects on cell infiltrates on bronchoalveolar lavages were evaluated 24 h after last cigarette smoke exposure. Air exposed, Roflumilast (5 mg/kg oral) treated animals as well as animals treated with the CXCR2 antagonist SCH527123 (10 mg/kg twice a day oral-total daily dose 20 mg/kg) serve as controls.

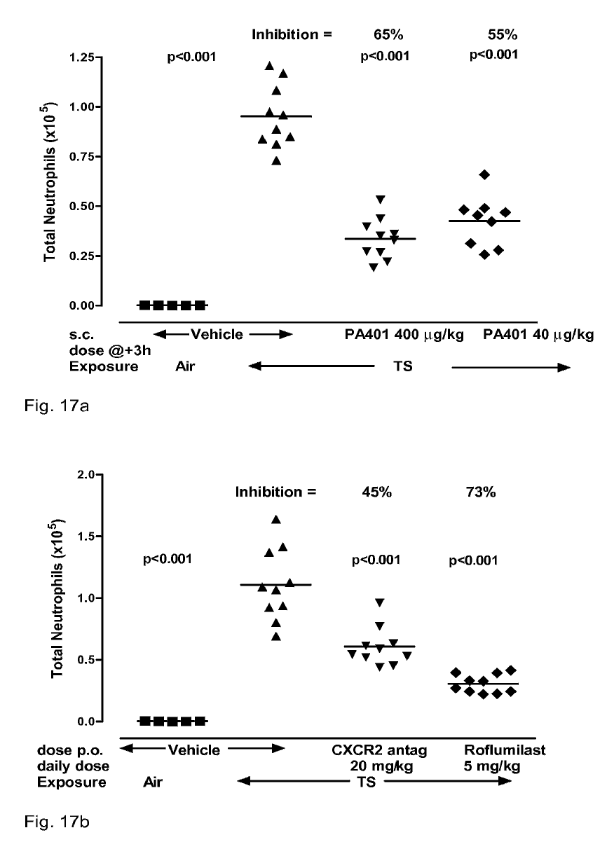

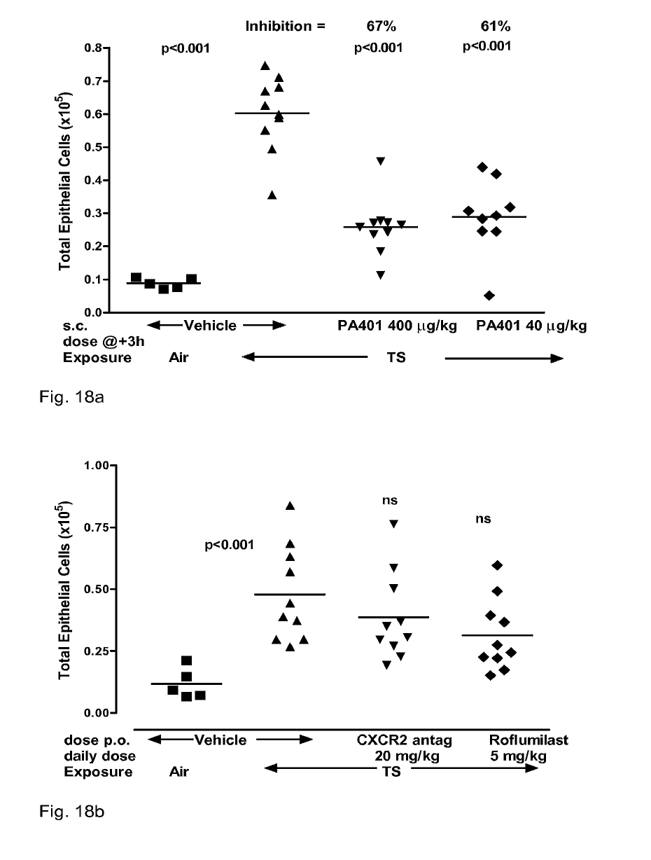

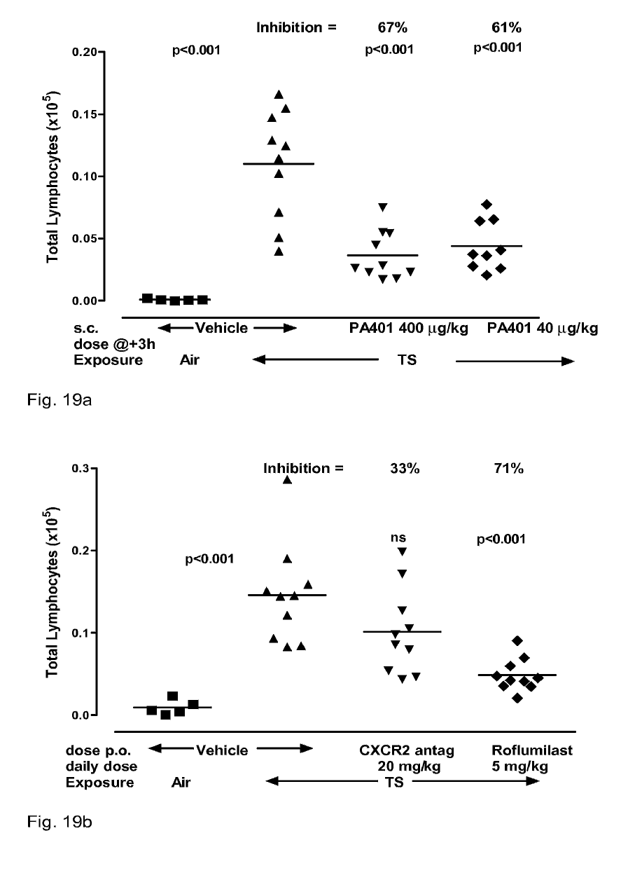

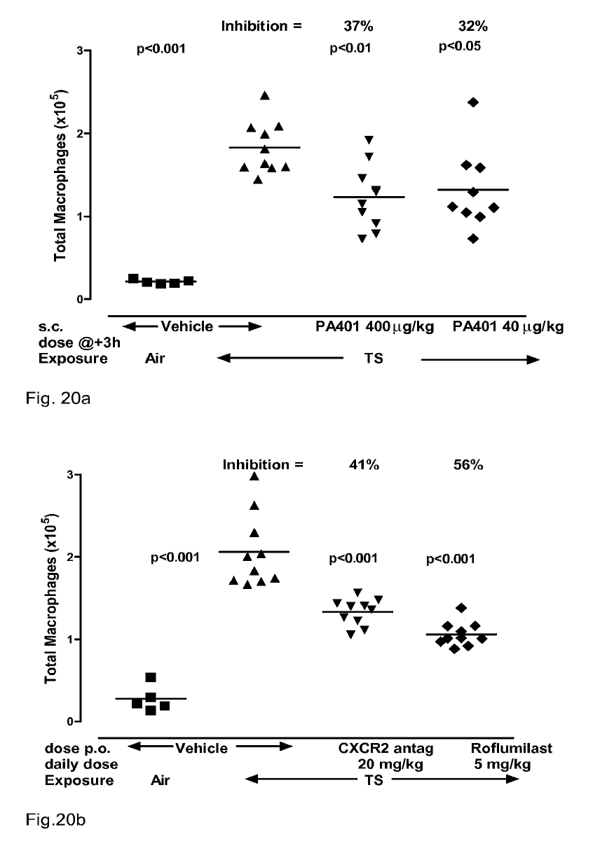

Significant effect of PA401on total cell infiltrates was observed after 400 and 40 μg/kg administration (FIG. 16). This was due to a significant reduction in neutrophil (FIG. 17), epithelial cells (FIG. 18), lymphocyte (FIG. 19) and macrophage (FIG. 20) numbers in BAL. The inhibitory effect of PA401 administrered at the dose of 400 μg/kg are comparable of that obtained with Roflumilast 5 mg/kg and the CXCR2 antagonist SCH527123 20 mg (10 mg/kg twice a day). This study was performed at Argenta Discovery Ltd, UK.

PA401

This study demonstrates activity of PA401 administered once a day subcutaneously at the doses of 400 μg/kg and 40 μg/kg on mixed cell infiltration induced by 11-day repeated exposure to cigarette smoke.

### Example 6

PA401 Effects in Lung Inflammation After Local Delivery to the Lung.

An experiment was performed to verify activity of PA401 after local delivery to the lung in an animal model of LPS induced lung inflammation.

In this experiment lung inflammation was induced by the delivery of LPS by aerosol (Salmonella enterica, 3.5 mg/7 mL over 30 min) in male Balb/c mice. PA401 was administered intra-tracheally (i.t.) using a MicroSprayer (FJM-250 syringe; PennCentury), a device that allows delivery of a plume of aerosol (mass median diameter 16-22 μm) directly into the lungs. PA401 i.t. administration was performed at the doses of 100, 40, 10 and 4 μg/kg either 1 h before LPS exposure, or 1 h after the end of LPS exposure. Bronchoalveolar lavage (BAL) was performed 8 h post LPS exposure and total cell and neutrophil numbers were measured. Dexamethasone 20 μg/20 μl mouse i.t. was used as reference compound.

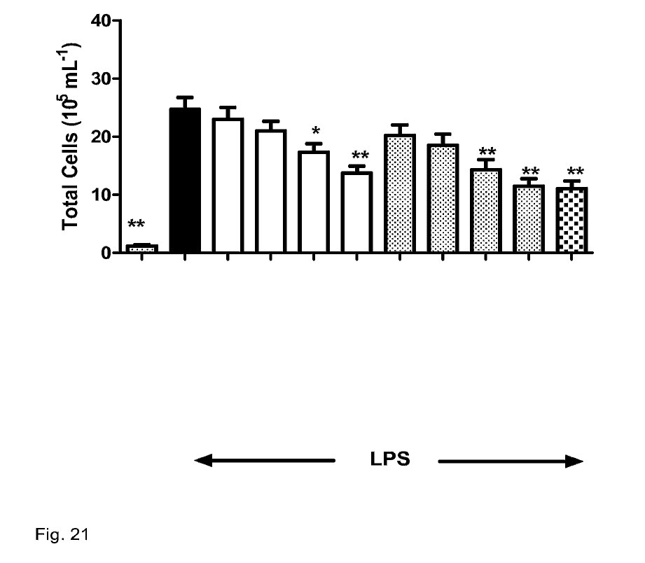

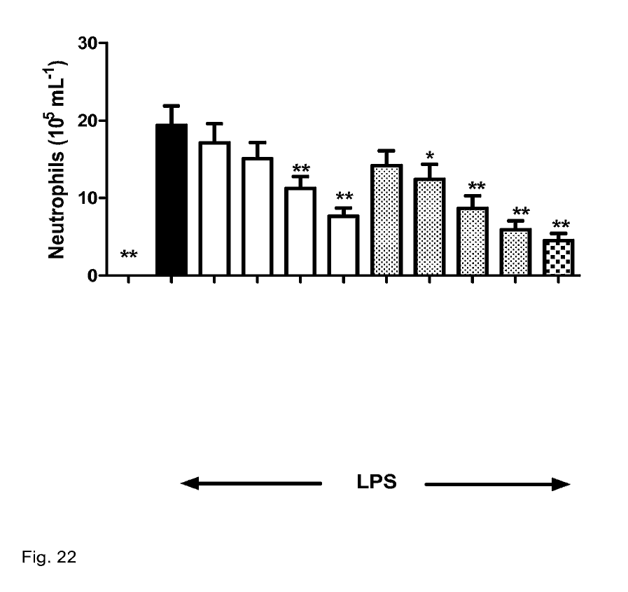

PA401 induced a significant dose dependent reduction of total cells in BAL (FIG. 21), mainly due to neutrophil number reduction (FIG. 22). The therapeutic treatment (t=+1 h) resulted in about 10% improved activity, compared to the prophylactic treatment (−1 h) with doses of 100, 40 and 10 μg/kg being significantly active.

FIG. 21 shows the dose-response activity on PA401 intra-tracheal administration 1 hour before (−1 h) and 1 hour after (+1 h)LPS aerosol exposure on total cell count in the bronchoalveolar lavage collected 8 hours post LPS. ANOVA followed by Dunnett's test: *p<0.05;**p<0.01 versus vehicle treated animals.

FIG. 22 shows the dose-response activity on PA401 intra-tracheal administration 1 hour before (−1 h) and 1 hour after (+1 h) LPS aerosol exposure on neutrophils count in the bronchoalveolar lavage collected 8 hours post LPS. ANOVA followed by Dunnett's test: *p<0.05;**p<0.01 versus vehicle treated animals.

These data demonstrate activity of PA401 following local delivery to the lung and open the possibility to the use of this administration route, which has normally good patient compliance, as alternative to intravenous or subcutaneous administration for chronic lung indications

### REFERENCES

Aaron S D, Angel J B, Lunau M, et al. Granulocyte inflammatory markers and airway infection during acute exacerbation of chronic obstructive pulmonary disease. Am J Respir Crit Care Med 2001; 163: 349-55.

Abraham E. Neutrophils and acute lung injury, Crit Cre Med 2003; Suppl: S195-S199.

Aggarwal A, baker C S, Evans T W et al. G-Csf and II-8 but not GM-CSF correlate with disease severity of pulmonary neutrophilia in acute respiratory distress syndrome. Eur Resp J 2000; 15: 895-901.

Anees W, Huggins V, Pavord I D, et al. Occupational asthma due to low molecular weight agents: eosinophilic and non-eosinophilic variants. Thorax 2002; 57:231-6.

Bernard G R, Artigas A, Brigham K L et al. The American-European Consensus Conference on ARDS. Definitions, mechanisms, relevant outcomes, and clinical trial coordination. Am j Respir Crit Care Med 1994; 149: 818-824.

Brennan S, Cooper D, Sly P D. Directed neutrophil migration to IL-8 is increased in cystic fibrosis: a study of the effect of erythromycin. Thorax 2001; 56: 62-64.

Bousquet J, Chanez P, Lacoste J Y, et al. Eosinophilic inflammation in asthma. N Engl J Med 1990; 323: 1033-9.

Chalmers G W, Macleod K J, Thomson L, et al. Smoking and airway inflammation in patients with mild asthma. Chest 2001; 120: 1917-22.

Chapman R W, Minnicozzi M, Celly C S, et al. A novel, orally active CXCR1/2 receptor antagonist, Sch527123, inhibits neutrophil recruitment, mucus production, and goblet cell hyperplasia in animal models of pulmonary inflammation. J Pharmacol Exp Ther 2007; 22(2): 486-93.

Chmiel J F, Berger M, Konstan M W. The role of inflammation in the patho-physiology of CF lung disease. Clin Rev Allergy Immunol 2002; 23(1): 5-27.

Cowburn S C, Condliffe A M, Farhai N et al. Advances in neutrophil biology. Clinical Implications. Chest 2008; 134: 606-612.

Culpitt S V, Maziak W, Luokidis S et al. Effects of high dose inhaled steroids on cells, cytokines, and proteases in induced sputum in chronic obstructive pulmonary disease. Am J Respir Crit Care Med 1999; 160: 1635-1639.

De Boer W I, Yao H, Rahman I. Future therapeutic treatment of COPD: struggle between antioxidants and cytokines. I J COPD 2007; 2(3): 205-228.

Donnelly S C, Strieter R M, Kunkel S L et al. Interleukine-8 and development of adult distress syndrome in at-risk patients groups. Lancet 1993; 341: 643-647.

Douwes J, Gibson P, Pekkanen J, et al. Non-eosinophilic asthma: importance and possible mechanisms. Thorax 2002; 57: 643-648

Fabbri L, Pauwels R A, Hurds S S. Global strategy for the diagnosis, management and prevention of chronic obstructive pulmonary disease: GOLD executive summary updated 2003. J COPD 2004; 1: 105-141.

Fahy J V, Kim K W, Liu J, et al. Prominent neutrophilic inflammation in sputum from subjects with asthma exacerbation. J Allergy Clin Immunol 1995; 95: 843-52.

Fahy J V. Eosinophilic and neutrophilic inflammation in asthma. Insight from Clinical studies. Proc Am Thorac Soc 2009; 6: 256-259.

Fitzgerald M F, Fox J C. Emerging trends in the therapy of COPD: novel anti-inflammatory agents in clinical development. Drug Discov Today 2007; 12(11/12): 479-485.

Frevert C W, Kinsella M G, Vathanaprida C et al. Binding of interleikin-8 to heparin and chondroitin sulfate in lung tissue. Am J Respir Cell Mol Biol 2003; 28: 464-472.

Green R H, Brightling C E, Woltmann G, et al. Analysis of induced sputum in adults with asthma: identification of subgroup with isolated sputum neutrophilia and poor response to inhaled corticosteroids. Thorax 2002; 57: 875-9.

Guerassimov A, Hoshino Y, Takubo Y et al. The development of emphysema in cigarette smoke-exposed mice is strain dependent. Am J Respir Crit Care Med 2004; 170. 974-980.

Kamath A V, Pavord I D, Ruparelia P R et al. Is the neutrophil the key effector cell in severe asthma? Thorax 2005; 60: 529-530.

Kharitonov S A, Sjöbring U. Lypopolysaccharide challenge in humans as a model for chronic obstructive lung disease exacerbations. Contrib Microbiol 2007; 14: 83-100.

Kim V, Rogers T J, Griner Gj. New concepts in the pathology of Chronic Obstructive Pulmonary Disese. Proc Am Thorac Soc 2008; 5: 478-485.

Kitz R, Rose M A, Placzek K et al. LPS inhalation challenge: a new tool to characterize the inflammatory response in humans. Med Microbiol Immunol 2008; 197: 13-19.

Koshika T, Ishizaka A, Nagatomi I et al. Pretreatment with FK506 improves survival rate and gas exchange in canine model of acute lung injury. Am J Respir Crit Care Med. 2001; 163 (1): 79-84.

Jatakanon A, Uasuf C, Maziak W, et al. Neutrophilic inflammation in severe persistent asthma. Am J Respir Crit Care Med 1999; 160: 1532-9.

Lamblin C, Gosset P, Tillie-Leblond I et al. Bronchial neutrophilia in patients with non infectious status asthmaticus. Am J Respir Crit Care Med 1998; 157: 394-402.

Lefant C, Khaltaev N. Workshop report: Global strategy for the diagnosis, management and prevention of COPD. URL: http://goldcopd.org/Guidelineitem.asp

Little S A, Macleod K J, Chalmers G W, et al. Association of forced expiratory volume with disease duration and sputum neutrophils in chronic asthma. Am J Med 2002; 112: 446-452.

Lopez A D, Murray C C. The global burden of disease, 1990-220. Nat Med 1998; 4: 1241-1243.

McDougall C M, Helmes P J. Neutrophil airway inflammation in childhood asthma. Thorax 2006; 61(9): 739-741.

Mahadeva R, Shapiro S D. Chronic obstructive pulmonary disease-3: experimental animal models of pulmonary emphysema. Thorax 2002; 57: 908-914.

Maris N A, de VOS A F, Dessing M C et al. Antiinflammatory effects of salmeterol after inhalation of lipopolysaccharide by healthy volunteers. Am J Respir Crit Care Med 2005; 172(7): 878-884.

Martin R J, Cicutto L C, Smith H R, et al. Airways inflammation in nocturnal asthma. Am Rev Respir Dis 1991; 143: 351-357.

Nakamura H, Yoshimura K, McElvaney N G, et al. Neutrophil elastase in respiratory epithelial lining fluid of individuals with cystic fibrosis induces interleukin-8 gene expression in a human bronchial epithelial cell line. J Clin Inves. 1992; 89: 1478-1484

Nocker R E, Schoonbrood D F, van de Graaf E A, et al. Interleukin-8 in airway inflammation in patients with asthma and chronic obstructive pulmonary disease. Int Arch Allergy Immunol 1996; 109: 183-191

Ordonez C L, Shaughnessy T E, Matthay M A, et al. Increased neutrophil numbers and IL-8 levels in airway secretions in acute severe asthma: clinical and biological significance. Am J Respir Crit Care Med 2000; 161: 1185-1190.

Pauwels R A, Buist As, Calverley P M, et al. Global strategy for the diagnosis, management, and prevention of chronic obstructive pulmonary disease. NHLBI/WHO Global Initiative for Chronic Obstructive Pulmonary Disease (GOLD) Workshop summary. Am J Respir Crit Care Med. 2005; 5: 1256-1276.

Peleman R A, Rytila P H, Kips J C et al. The cellular composition of induced sputum in chronic obstructive pulmonary disease. Eur Respir J 1999; 13: 839-843.

Puha J, Badia J R, Adhikari et al. Has mortality from acute respiratory distress syndrome decreased over time?: A systematic review. Am J Respir Crit Care Med 2009; 179(3): 220-227.

Ratjen F A. Diagnosing and managing infection in CF. Paediatr Respir rev 2006; 7 (suppl1): S151-S153.

Ratjen F A. Cystic fibrosis: pathogenesis and future treatment strategies. Respir Care 2009; 54(5): 595-602.

Reid P T, Donnelly Sc, Haslett C. Inflammatory predictors for the development of adult respiratory distress syndrome. Thorax 1995; 50: 10023-10026.

Reutershan J, Basit A, Galkina E V et al. Sequential recruitment of neutrophils into lung and bronchoalveolar lavage fluid in LPS-induced acute lung injury. Am J Physiol Cell Mol Physiol Lung Cell Mol Physiol 2005; 289 (5): L807-L815.

Riordan J R, Rommens J M, Kerem B, et al. Identification of the cystic fibrosis gene: cloning and characterization of complementary DNA. Science. 1989; 245 :1066-1073.

Sagel S D, Accurso F J. Monitoring inflammation in CF. Cytokines. Clin Rev Allergy Immunol. 2002; 23:41-57.

Shaw D E, Berry M A, Hargadon B et al. Association between neutrophilic airway inflammation and airflow limitation in adults with asthma. Chest 2007; 132: 1871-1875.

Smith L S, Kajikawa O, Elson G et al. effect of Toll-like receptor 4 blockade on pulmonary inflammation caused by mechanical ventilation and bacterial endotoxin. Exp Lung Res 2008; 34: 225-245.

Souza-Fernandes A B, Pelosi P, Rocco P R M. Bench-to-bedside review: the role of glycosaminoglycans in respiratory disease. Crit care 2006; 10(6): 237-252.

Sparrow D, Glynn R J, Cohen M et al. The relationship of the peripheral leukocyte count and cigarette smiking to pulmonary function among adult men. Chest 1984. 86: 383-386.

Spruit M A, Gosselink R, Troosters T, et al. Muscle force during an acute exacerbation in hospitalized patients with COPD and its relationship with CXCL8 and IGF-I. Thorax 2003; 58: 752-756.

Sur S, Crotty T B, Kephart G M, et al. Sudden-onset fatal asthma: a distinct entity with few eosinophils and relatively more neutrophils in the airway submucosa? Am Rev Respir Dis 1993; 148: 713-719.

Tabary O, Zahm J M, Hinnrasky J, et al. Selective up-regulation of chemokine IL-8 expression in cystic fibrosis bronchial gland cells in vivo and in vitro. Am J Pathol 1998; 153: 921-930.

Tabary O, Corvol H, Boncoeur E et al. Adherence of airway neuthrophils and inflammatory response are increased in Cf airway epithelial cell-neutrophil interactions. Am J Physiol Lung Cell Mol Physiol 2006; 290 (3): L588-596.

Tanino M, Betsuyaku T, Takeyabu K, et al. Increased levels of interleukin-8 in BAL fluid from smokers susceptible to pulmonary emphysema. Thorax 2002; 57: 405-411

Van den Hoven R, Duvigneau J C, Hertl R T et al. Clenbuterol affects the expression of messenger RNA for interleukin 10 in peripheral leukocytes from horses challenged intrabronchially with lipopolysaccharides. Vet Res Commun 2006; 30(8): 921-928.

Vlahos R, Bozinovski S, Jones J E et al. Differential protease, innate immunity, and NK-kB induction profiles during lung inflammation induced by subchronic cigarette smoke exposure in mice. Am J Physiol Lung Cell Mol Physiol 2006; 290: L931-L945.

Wenzel S E, Schwartz L B, Langmack E L, et al. Evidence that severe asthma can be divided pathologically into two inflammatory subtypes with distinct physiologic and clinical characteristics. Am J Respir Crit Care Med 1999; 160: 1001-8.

Ware L B, Matthay M A. The acute respiratory distress syndrome. N Engl J Med 2000; 342: 1334-1349.

Waerhaug K, Kuzkov W, Kulin V N et al. Inhaled aerosolized recombinant human protein C ameliorates endotoxin-induced lung injury in anesthetized sheep. Crit Care 2009; 13(2) R51.

Wenzel S E, Szefler S J, Leung D Y M, et al. Bronchoscopic evaluation of severe asthma: persistent inflammation associated with high dose glucocorticoids. Am J Respir Crit Care Med 1997; 156: 737-473.

Woolhouse I S, Bayley D L, Stockley R A. Sputum chemotactic activity in chronic obstructive pulmonary disease. Effects of a1-antitrypsin deficiency and the role of leukotriene B4 and interleukine 8. Thorax 2002; 57: 709-714.

Wu Y, Singer M, Thouron F et al. Effect of surfactant on pulmonary expression of type IIA PLA(2) in an animal model of acute lung injury. Am J Physiol Lung Cell Mol Physiol 2002; 282(4): L743-750.

Yamamoto C, Yoneda T, Yoshikawa M, et al. Airway inflammation in COPD assessed by sputum levels of interleukin-8. Chest 1997; 112: 505-510.

The ENFUMOSA study group. The ENFUMOSA cross-sectional European multicentre study of the clinical phenotype of severe asthma. Eur Respir J 2003; 22-470-477.

1. Modified interleukin 8 (IL-8) having increased glyosaminoglycan (GAG) binding affinity and further inhibited or down-regulated G-protein coupled receptor (GPCR) activity compared to the respective wild type IL-8 for use in the prevention or treatment of lung inflammation with neutrophilic infiltration.

2. Modified IL-8 according to claim 1, wherein the modified IL-8 comprises a GAG binding region which is modified by substitution, insertion, and/or deletion of at least one amino acid in order to increase the relative amount of basic amino acids in said GAG binding region, and/or reduce the amount of bulky and/or acidic amino acids in said GAG binding region preferably at a solvent exposed position.

3. Modified IL-8 according to claim 2, wherein at least one amino acid selected from the group consisting of Arg, Lys, and His is inserted into said GAG binding region.

4. Modified IL-8 according to claim 1, wherein positions 17, 21, 70, and/or 71 of IL-8 are substituted by Arg, Lys, His, Asn and/or Gln.

5. Modified IL-8 according to claim 1, wherein the GPCR binding region of IL-8 is modified by deletion, insertion, and/or substitution, preferably with alanine, a sterically and/or electrostatically similar residue.

6. Modified IL-8 according to claim 1 wherein the amino acid sequence of the modified IL-8 molecule is
(SEQ ID No. 2)
(X1)n(X2)m KTYSKP(X3)HPK (X4)IKELRVIES GPHCANTEII
VKLSDGRELC LDPKENWVQR VVEKFLKRA(X5) (X6)S
wherein X1 is of amino acid sequence SAKELR,
wherein X2 is of amino acid sequence CQCI,
wherein X3 is selected of the group consisting of F, R, K, H, N and/or Q, preferably X3is K,
wherein X4 is selected of the group consisting of F, R, K, H, N and/or Q, preferably X4 is K
wherein X5 is selected of the group consisting of E, R, K, H, N and/or Q, preferably X5 is K,
wherein X6 is selected of the group consisting of R, K, H, N and/or Q, preferably X6 is K,
and wherein n and/or m is 0 or 1.

7. Modified IL-8 according to claim 1, wherein said modified IL-8 molecule is selected from the group consisting of del6F17RE70KN71R, del6F17RE70RN71K, del6E70KN71K, and del6F17KF21KE70KN71K.

8. Modified IL-8 according to claim 1, wherein the amino acid sequence of the modified IL-8 molecule is CQCI KTY SKPKHPKKIK ELRVIESGPH CANTEIIVKL SDGRELCLDP KENWVQRVVE KFLKRAKKS (SEQ ID No. 1).

9. Modified IL-8 according to claim 1, wherein the lung inflammation with neutrophilic infiltration is selected from chronic obstructive pulmonary disease, cystic fibrosis, severe asthma, bronchitis, broncheolitis, acute lung injury and acute respiratory distress syndrome.

10. Modified IL-8 according to claim 1 formulated as inhalant.

11. Method for treatment of lung inflammation with neutrophilic infiltration in a subject in need thereof comprising administering to the subject a therapeutically effective amount of modified IL-8.

12. Method according to claim 11 wherein the administration is by inhalation or by intratracheal administration.
